(19) United States
(12) Patent Application Publication
(10) Pub. No.: US 2005/0171102 A1
(43) Pub. Date: Aug. 4, 2005

(54) Semicarbazide derivatives for combating thromboembolic diseases

(76) Inventors: Werner Mederski, Zwingenberg (DE); Dieter Dorsch, Ober-Ramstadt (DE); Christos Tsaklakidis, Weinheim (DE); Christopher Barnes, Bad Soden (DE); Bertram Cezanne, Morfelden-Walldorf (DE); Johannes Gleitz, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/513,451

(22) PCT Filed: Apr. 7, 2003
(86) PCT No.: PCT/EP03/03581

(30) Foreign Application Priority Data
May 4, 2002 (DE) 102 20 048.3

(51) Int. Cl. C07D 413/02; A61K 31/5377; A61K 31/501; A61K 31/496; A61K 31/454; A61K 31/4245

(52) U.S. Cl. 514/235.5; 514/364; 514/254.03; 514/326; 514/252.05; 544/238; 544/138; 544/367; 546/209

(57) ABSTRACT

Novel compounds of the formula I in which R, R¹, R¹, X are as defined in Patent claim 1, are inhibitors of coagulation factor Xa and can be employed for the prophylaxis and/or therapy of thromboembolic diseases and for the treatment of tumours.

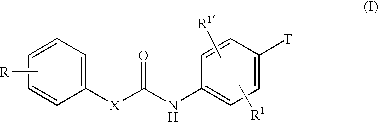

The invention relates to compounds of the formula I 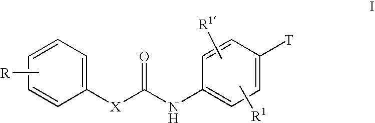
in which
R denotes C(═NH)—NH₂, which may also be monosubstituted by OH, COA or OCOOA,
   or is CH₂NH₂, CONH₂, CN, 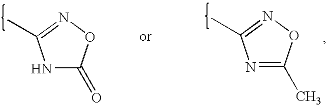
denotes —N(Ar)—NH— or —NH—N(Ar)—,
R¹, R¹′ each, independently of one another, denote H, A, OH, OA, —O(C═O)A, Hal, CF₃, CN, COOH, COOA, CH₂NH₂, NH₂, NHA or NA₂,
T denotes a monocyclic or bicyclic saturated or unsaturated heterocyclic ring having from 1 to 4 N, O and/or S atoms, which may be mono-, di- or trisubstituted by carbonyl oxygen and/or OH, Hal or A,
Ar denotes phenyl which is unsubstituted or mono-, di- or trisubstituted by A, OH, OA, —O—(C═O)-A, Hal, NH₂, NHA, NA₂, NO₂, CF₃, CN, COA, COOH, COOA, CONH₂ or CH₂NH₂,
A denotes unbranched, branched or cyclic alkyl having 1-10 carbon atoms,
Hal denotes F, Cl, Br or I,
   and pharmaceutically usable derivatives, solvates and stereoisomers thereof, including mixtures thereof in all ratios.

The invention had the object of finding novel compounds having valuable properties, in particular those which can be used for the preparation of medicaments.

It has been found that the compounds of the formula I and salts thereof have very valuable pharmacological properties and are well tolerated. In particular, they exhibit factor Xa-inhibiting properties and can therefore be employed for combating and preventing thromboembolic diseases, such as thromboses, myocardial infarction, arteriosclerosis, inflammation, apoplexy, angina pectoris, restenosis after angioplasty and claudicatio intermittens.

The compounds of the formula I according to the invention may furthermore be inhibitors of the coagulation factors factor VIIa, factor IXa and thrombin in the blood coagulation cascade.

Aromatic amidine derivatives having an antithrombotic action are disclosed, for example, in EP 0 540 051 B1, WO 98/28269, WO 00/71508, WO 00/71511, WO 00/71493, WO 00/71507, WO 00/71509, WO 00/71512, WO 00/71515 or WO 00/71516. cyclic guanidines for the treatment of thromboembolic diseases are described, for example, in WO 97/08165. Aromatic heterocyclic compounds having a factor Xa inhibitory activity are disclosed, for example, in WO 96/10022. Substituted N-[(aminoiminomethyl)phenylalkyl]azaheterocyclylamides as factor Xa inhibitors are described in WO 96/40679.

The antithrombotic and anticoagulant effect of the compounds according to the invention is attributed to the inhibitory action against activated coagulation protease, known by the name factor Xa, or to the inhibition of other activated serine proteases, such as factor VIIa, factor IXa or thrombin.

Factor Xa is one of the proteases involved in the complex process of blood coagulation. Factor Xa catalyses the conversion of prothrombin into thrombin. Thrombin cleaves fibrinogen into fibrin monomers, which, after crosslinking, make an elementary contribution to thrombus formation. Activation of thrombin may result in the occurrence of thromboembolic diseases. However, inhibition of thrombin may inhibit the fibrin formation involved in thrombus formation.

The inhibition of thrombin can be measured, for example by the method of G. F. Cousins et al. in Circulation 1996, 94, 1705-1712.

Inhibition of factor Xa can thus prevent the formation of thrombin. The compounds of the formula I according to the invention and salts thereof engage in the blood coagulation process by inhibiting factor Xa and thus inhibit the formation of thrombi.

The inhibition of factor Xa by the compounds according to the invention and the measurement of the anticoagulant and antithrombotic activity can be determined by conventional in-vitro or in-vivo methods. A suitable method is described, for example, by J. Hauptmann et al. in Thrombosis and Haemostasis 1990, 63, 220-223.

The inhibition of factor Xa can be measured, for example by the method of T. Hara et al. in Thromb. Haemostas. 1994, 71, 314-319.

Coagulation factor VIIa initiates the extrinsic part of the coagulation cascade after binding to tissue factor and contributes to the activation of factor X to give factor Xa. Inhibition of factor VIIa thus prevents the formation of factor Xa and thus subsequent thrombin formation.

The inhibition of factor VIIa by the compounds according to the invention and the measurement of the anticoagulant and antithrombotic activity can be determined by conventional in-vitro or in-vivo methods. A conventional method for the measurement of the inhibition of factor VIIa is described, for example, by H. F. Ronning et al. in Thrombosis Research 1996, 84, 73-81.

Coagulation factor IXa is generated in the intrinsic coagulation cascade and is likewise involved in the activation of factor X to give factor Xa. Inhibition of factor IXa can therefore prevent the formation of factor Xa in a different way.

The inhibition of factor IXa by the compounds according to the invention and the measurement of the anticoagulant and antithrombotic activity can be determined by conventional in-vitro or in-vivo methods. A suitable method is described, for example, by J. Chang et al. in Journal of Biological Chemistry 1998, 273, 12089-12094.

The compounds according to the invention may furthermore be used for the treatment of tumours, tumour diseases and/or tumour metastases. A correlation between tissue factor TF/factor VIIa and the development of various types of cancer has been indicated by T. Taniguchi and N. R. Lemoine in Biomed. Health Res. (2000), 41 (Molecular Pathogenesis of Pancreatic Cancer), 57-59.

The publications listed below describe an antitumoral action of TF-VII and factor Xa inhibitors for various types of tumour:
K. M. Donnelly et al. in Thromb. Haemost. 1998; 79: 1041-1047;
E. G. Fischer et al. in J. Clin. Invest. 104: 1213-1221 (1999);
B. M. Mueller et al. in J. Clin. Invest. 101: 1372-1378 (1998);
M. E. Bromberg et al. in Thromb. Haemost. 1999; 82: 88-92

The compounds of the formula I can be employed as medicament active ingredients in human and veterinary medicine, in particular for the treatment and prevention of thromboembolic diseases, such as thromboses, myocardial infarction, arteriosclerosis, inflammation, apoplexy, angina pectoris, restenosis after angioplasty, claudicatio intermittens, venous thrombosis, pulmonary embolism, arterial thrombosis, myocardial ischaemia, unstable angina and strokes based on thrombosis.

The compounds according to the invention are also employed for the treatment or prophylaxis of atherosclerotic diseases, such as coronary arterial disease, cerebral arterial disease or peripheral arterial disease. The compounds are also employed in combination with other thrombolytic agents in myocardial infarction, furthermore for prophylaxis for reocclusion after thrombolysis, percutaneous transluminal angioplasty (PTCA) and coronary bypass operations.

The compounds according to the invention are furthermore used for the prevention of rethrombosis in microsurgery, furthermore as anticoagulants in connection with artificial organs or in haemodialysis.

The compounds are furthermore used in the cleaning of catheters and medical aids in patients in vivo, or as anticoagulants for the preservation of blood, plasma and other blood products in vitro. The compounds according to the invention are furthermore used for diseases in which blood coagulation makes a crucial contribution towards the course of the disease or represents a source of secondary pathology, such as, for example, in cancer, including metastasis, inflammatory diseases, including arthritis, and diabetes.

The compounds according to the invention are furthermore used for the treatment of migraine (F. Morales-Asin et al., Headache, 40, 2000, 45-47).

In the treatment of the diseases described, the compounds according to the invention are also employed in combination with other thrombolytically active compounds, such as, for example, with the “tissue plasminogen activator” t-PA, modified t-PA, streptokinase or urokinase. The compounds according to the invention are administered either at the same time as or before or after the other substances mentioned.

Particular preference is given to simultaneous administration with aspirin in order to prevent recurrence of the clot formation.

The compounds according to the invention are also used in combination with blood platelet glycoprotein receptor (IIb/IIIa) antagonists, which inhibit blood platelet aggregation.

The invention relates to the compounds of the formula I and salts thereof and to a process for the preparation of compounds of the formula I according to claims 1-9 and pharmaceutically usable derivatives, solvates and stereoisomers thereof, characterised in that
a) they are liberated from one of their functional derivatives by treatment with a solvolysing and/or hydrogenolysing agent by
i) liberating an amidino group from the oxadiazole derivative or oxazolidinone derivative thereof by hydrogenolysis or solvolysis,
ii) replacing a conventional amino-protecting group with hydrogen by treatment with a solvolysing or hydrogenolysing agent or liberating an amino group protected by a conventional protecting group,
b) a radical R is converted into another radical R by
   i) converting a cyano group into an amidino group,
   ii) reducing a cyano group to an aminoalkyl group,
      and/or a base or acid of the formula I is converted into one of its salts.

The invention also relates to the optically active forms (stereoisomers), the enantiomers, the racemates, the diastereomers and the hydrates and solvates of these compounds. The term solvates of the compounds is taken to mean adductions of inert solvent molecules onto the compounds which form owing to their mutual attractive force. Solvates are, for example, mono- or dihydrates or alcoholates.

The term pharmaceutically usable derivatives is taken to mean, for example, the salts of the compounds according to the invention and so-called prodrug compounds.

The term prodrug derivatives is taken to mean compounds of the formula I which have been modified with, for example, alkyl or acyl groups, sugars or oligopeptides and which are rapidly cleaved in the organism to form the active compounds according to the invention.

These also include biodegradable polymer derivatives of the compounds according to the invention, as described, for example, in Int. J. Pharm. 115, 61-67 (1995).

The invention also relates to mixtures of the compounds of the formula I according to the invention, for example mixtures of two diastereomers, for example in the ratio 1:1, 1:2, 1:3, 1:4, 1:5, 1:10, 1:100 or 1:1000.

These are particularly preferably mixtures of stereoisomeric compounds.

For all radicals which occur more than once, such as, for example, A, their meanings are independent of one another.

Above and below, the radicals and parameters R, R¹, R^{1′}, X and T are as defined under the formula I, unless expressly indicated otherwise.

A denotes alkyl, is unbranched (linear), branched or cyclic, and has 1, 2, 3, 4, 5, 6, 7, 8, 9 or 10 carbon atoms. A preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl.

A very particularly preferably denotes alkyl having 1, 2, 3, 4, 5 or 6 carbon atoms, preferably methyl, ethyl, propyl, isopropyl, butyl, isobutyl, sec-butyl, tert-butyl, pentyl, hexyl, cyclopentyl, cyclohexyl, trifluoromethyl, pentafluoroethyl or 1,1,1-trifluoroethyl.

Cyclic alkyl preferably denotes cyclopropyl, cyclobutyl, cyclopentyl, cyclohexyl or cycloheptyl.

—COA (acyl) preferably denotes acetyl, propionyl, furthermore also butyryl, pentanoyl, hexanoyl or, for example, benzoyl.

Hal preferably denotes F, Cl or Br, but alternatively I.

The invention also relates, in particular, to the —C(═NH)—NH₂ compounds of the formula I which are substituted by —COA, —COOA, —OH or by a conventional amino-protecting group.

R preferably denotes amidino, which may also be substituted by OH, or is CH₂NH₂.

R¹ preferably denotes H, F, Cl or A, for example preferably CH₃ or CF₃.

R^{1′} preferably denotes H.

Ar denotes, for example, unsubstituted phenyl, furthermore preferably phenyl which is monosubstituted, disubstituted or trisubstituted, for example, by A, fluorine, chlorine, hydroxyl, methoxy, ethoxy, propoxy, butoxy, pentyloxy, hexyloxy, nitro, cyano, formyl, acetyl, propionyl, trifluoromethyl, amino, methylamino, ethylamino, dimethylamino, diethylamino, carboxyl, methoxycarbonyl, ethoxycarbonyl or by aminocarbonyl.

Ar very particularly preferably denotes phenyl which is unsubstituted or monosubstituted by Cl or F.

T preferably denotes a monocyclic saturated or unsaturated heterocyclic ring having 1 to 2 N and/or O atoms, which may be monosubstituted or disubstituted by carbonyl oxygen, OH or OA.

T particularly preferably denotes, for example, piperidin-1-yl, 2-oxopiperidin-1-yl, 2-oxopyrrolidin-1-yl, pyrrolidin-1-yl, 2-oxo-1H-pyridin-1-yl, 3-oxomorpholin-4-yl, morpholin-4-yl, 4-oxo-1H-pyridin-1-yl, 2,6-dioxopiperidin-1-yl, 2-oxopiperazin-1-yl, 2,6-dioxopiperazin-1-yl, 2,5-dioxopyrrolidin-1-yl, 2-oxo-1,3-oxazolidin-3-yl, 3-oxo-2H-pyridazin-2-yl, 2-caprolactam-1-yl (=2-oxoazepan-1-yl), 2-hydroxy-6-oxopiperazin-1-yl, 2-methoxy-6-oxopiperazin-1-yl, 2-azabicyclo[2.2.2]octan-3-on-2-yl, 5,6-dihydro-1H-pyrimidin-2-oxo-1-yl or 4H-1,4-oxazin-4-yl.

Very particular preference is given to 2-oxopiperidin-1-yl, 2-oxopyrrolidin-1-yl, 3-oxomorpholin-4-yl, morpholin-4-yl, piperidin-1-yl, pyrrolidin-1-yl or 3-oxo-2H-pyridazin-2-yl.

The compounds of the formula I may have one or more chiral centres and therefore occur in various stereoisomeric forms. The formula I covers all these forms.

Accordingly, the invention relates in particular to the compounds of the formula I in which at least one of the said radicals has one of the preferred meanings indicated above. Some preferred groups of compounds may be expressed by the following sub-formulae Ia to Ij, which conform to the formula I and in which the radicals not designated in greater detail are as defined under the formula I, but in which
in Ia R denotes amidino, which may also be substituted by OH, or denotes CH₂NH₂;
in Ib R¹ denotes H, A, CF₃ or Hal, and
   R^{1′} denotes H;
in Ic Ar denotes phenyl which is unsubstituted or monosubstituted by Cl or F;
in Id T denotes a monocyclic or bicyclic saturated or unsaturated heterocyclic ring having 1 to 2 N and/or O atoms, which may be monosubstituted or disubstituted by carbonyl oxygen and/or OH, Hal or A;
in Ie T denotes a monocyclic saturated or unsaturated heterocyclic ring having 1 or 2 N and/or O atoms, which may be monosubstituted or disubstituted by carbonyl oxygen;
in If T denotes piperidin-1-yl, 2-oxopiperidin-1-yl, pyrrolidin-1-yl, 2-oxopyrrolidin-1-yl, 2-oxo-1H-pyridin-1-yl, 3-oxomorpholin-4-yl, morpholin-4-yl, 4-oxo-1H-pyridin-1-yl, 2,6-dioxopiperidin-1-yl, 2-oxopiperazin-1-yl, 2,6-dioxopiperazin-1-yl, 2,5-dioxopyrrolidin-1-yl, 2-oxo-1,3-oxazolidin-3-yl, 3-oxo-2H-pyridazin-2-yl, 2-caprolactam-1-yl (=2-oxoazepan-1-yl), 2-hydroxy-6-oxopiperazin-1-yl, 2-azabicyclo[2.2.2]-octan-3-on-2-yl, 2-methoxy-6-oxopiperazin-1-yl, 5,6-dihydro-1H-pyrimidin-2-oxo-1-yl or 4H-1,4-oxazin-4-yl;
in Ig T denotes 2-oxopiperidin-1-yl, 2-oxopyrrolidin-1-yl, 3-oxomorpholin-4-yl, morpholin-4-yl, piperidin-1-yl, pyrrolidin-1-yl or 3-oxo-2H-pyridazin-2-yl;
in Ih R denotes amidino, which may also be substituted by OH, or denotes CH₂NH₂,
   R¹ denotes H, A, CF₃ or Hal,
   R^{1′} denotes H,
   Ar denotes phenyl which is unsubstituted or monosubstituted by Cl or F,
   T denotes 2-oxopiperidin-1-yl, 2-oxopyrrolidin-1-yl, 3-oxomorpholin-4-yl, morpholin-4-yl, piperidin-1-yl, pyrrolidin-1-yl or 3-oxo-2H-pyridazin-2-yl;
in Ii R denotes amidino, which may also be substituted by OH, or denotes CN or CH₂NH₂,
   R¹ denotes H, A, CF₃ or Hal,
   R^{1′} denotes H,
   Ar denotes phenyl which is unsubstituted or monosubstituted by Cl or F,
   T denotes 2-oxopiperidin-1-yl, 2-oxopyrrolidin-1-yl, 3-oxomorpholin-4-yl, morpholin-4-yl, piperidin-1-yl, pyrrolidin-1-yl or 3-oxo-2H-pyridazin-2-yl;
in Ij R denotes amidino, which may also be substituted by OH, or denotes CN or CH₂NH₂,
   R¹ denotes H, A, CF₃ or Hal,
   R^{1′} denotes H or CF₃,
   Ar denotes phenyl which is unsubstituted or monosubstituted by Cl or F,
   T denotes 2-oxopiperidin-1-yl, 2-oxopyrrolidin-1-yl, 3-oxomorpholin-4-yl, morpholin-4-yl, piperidin-1-yl, pyrrolidin-1-yl or 3-oxo-2H-pyridazin-2-yl;
      and pharmaceutically usable derivatives, solvates and stereoisomers thereof, including mixtures thereof in all ratios.

The compounds of the formula I and also the starting materials for the preparation thereof are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

If desired, the starting materials can also be formed in situ so that they are not isolated from the reaction mixture, but instead are immediately converted further into the compounds of the formula I.

Compounds of the formula I can preferably be obtained by liberating compounds of the formula I from one of their functional derivatives by treatment with a solvolysing or hydrogenolysing agent.

Preferred starting materials for the solvolysis or hydrogenolysis are those which conform to the formula I, but contain corresponding protected amino and/or hydroxyl groups instead of one or more free amino and/or hydroxyl groups, preferably those which carry an amino-protecting group instead of an H atom bonded to an N atom, in particular those which carry an R′—N group, in which R′ denotes an amino-protecting group, instead of an HN group, and/or those which carry a hydroxyl-protecting group instead of the H atom of a hydroxyl group, for example those which conform to the formula I, but carry a —COOR″ group, in which R″ denotes a hydroxyl-protecting group, instead of a —COOH group.

Preferred starting materials are also the oxadiazole derivatives, which can be converted into the corresponding amidino compounds.

The amidino group can be liberated from the oxadiazole derivative thereof by, for example, treatment with hydrogen in the presence of a catalyst (for example Raney nickel). Suitable solvents are those indicated below, in particular alcohols, such as methanol or ethanol, organic acids, such as acetic acid or propionic acid, or mixtures thereof. The hydrogenolysis is generally carried out at temperatures between about 0 and 1000 and pressures between about 1 and 200 bar, preferably at 20-300 (room temperature) and 1-10 bar.

The oxadiazole group is introduced, for example, by reaction of the cyano compounds with hydroxylamine and reaction with phosgene, dialkyl carbonate, chloroformic acid esters, N,N′-carbonyldiimidazole or acetic anhydride.

It is also possible for a plurality of—identical or different—protected amino and/or hydroxyl groups to be present in the molecule of the starting material. If the protecting groups present are different from one another, they can in many cases be cleaved off selectively.

The term “amino-protecting group” is known in general terms and relates to groups which are suitable for protecting (blocking) an amino group against chemical reactions, but which can easily be removed after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their type and size is furthermore not crucial; however, preference is given to those having 1-20, in particular 1-8, carbon atoms. The term “acyl group” is to be understood in the broadest sense in connection with the present process. It includes acyl groups derived from aliphatic, araliphatic, aromatic or heterocyclic carboxylic acids or sulfonic acids, and, in particular, alkoxycarbonyl, aryloxycarbonyl and especially aralkoxycarbonyl groups. Examples of such acyl groups are alkanoyl, such as acetyl, propionyl, butyryl; aralkanoyl, such as phenylacetyl; aroyl, such as benzoyl or tolyl; aryloxyalkanoyl, such as POA; alkoxycarbonyl, such as methoxycarbonyl, ethoxycarbonyl, 2,2,2-trichloroethoxycarbonyl, BOC (tert-butoxycarbonyl), 2-iodoethoxycarbonyl; aralkoxycarbonyl, such as CBZ (“carbobenzoxy”), 4-methoxybenzyloxycarbonyl, FMOC; arylsulfonyl, such as Mtr. Preferred amino-protecting groups are BOC and Mtr, furthermore CBZ, Fmoc, benzyl and acetyl.

The term “hydroxyl-protecting group” is likewise known in general terms and relates to groups which are suitable for protecting a hydroxyl group against chemical reactions, but can easily be removed after the desired chemical reaction has been carried out elsewhere in the molecule. Typical of such groups are the above-mentioned unsubstituted or substituted aryl, aralkyl or acyl groups, furthermore also alkyl groups. The nature and size of the hydroxyl-protecting groups is not crucial since they are removed again after the desired chemical reaction or reaction sequence; preference is given to groups having 1-20, in particular 1-10, carbon atoms. Examples of hydroxyl-protecting groups are, inter alia, benzyl, 4-methoxybenzyl, p-nitrobenzoyl, p-toluenesulfonyl, tert-butyl and acetyl, where benzyl and tert-butyl are particularly preferred.

The compounds of the formula I are liberated from their functional derivatives—depending on the protecting group used—for example using strong acids, advantageously using TFA or perchloric acid, but also using other strong inorganic acids, such as hydrochloric acid or sulfuric acid, strong organic carboxylic acids, such as trichloroacetic acid, or sulfonic acids, such as benzene- or p-toluenesulfonic acid. The presence of an additional inert solvent is possible, but is not always necessary. Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water. Mixtures of the above-mentioned solvents are furthermore suitable. TFA is preferably used in excess without addition of a further solvent, perchloric acid is preferably used in the form of a mixture of acetic acid and 70% perchloric acid in the ratio 9:1. The reaction temperatures for the cleavage are advantageously between about 0 and about 50°, preferably between 15 and 30° (room temperature).

The BOC, OBut and Mtr groups can, for example, preferably be cleaved off using TFA in dichloromethane or using approximately 3 to 5N HCl in dioxane at 15-30°, the FMOC group can be cleaved off using an approximately 5 to 50% solution of dimethylamine, diethylamine or piperidine in DMF at 15-30°.

Protecting groups which can be removed hydrogenolytically (for example CBZ, benzyl or the liberation of the amidino group from the oxadiazole derivative thereof) can be cleaved off, for example, by treatment with hydrogen in the presence of a catalyst (for example a noble-metal catalyst, such as palladium, advantageously on a support, such as carbon). Suitable solvents here are those indicated above, in particular, for example, alcohols, such as methanol or ethanol, or amides, such as DMF. The hydrogenolysis is generally carried out at temperatures between about 0 and 1000 and pressures between about 1 and 200 bar, preferably at 20-30° and 1-10 bar. Hydrogenolysis of the CBZ group succeeds well, for example, on 5 to 10% Pd/C in methanol or using ammonium formate (instead of hydrogen) on Pd/C in methanol/DMF at 20-30°.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, trifluoromethylbenzene, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide, N-methylpyrrolidone (NMP) or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

A cyano group is converted into an amidino group by reaction with, for example, hydroxylamine followed by reduction of the N-hydroxyamidine using hydrogen in the presence of a catalyst, such as, for example, Pd/C. In order to prepare an amidine of the formula I, it is also possible to adduct ammonia onto a nitrile. The adduction is preferably carried out in a number of steps by, in a manner known per se, a) converting the nitrile into a thioamide using H₂S, converting the thioamide into the corresponding S-alkylimidothioester using an alkylating agent, for example CH₃I, and reacting the thioester in turn with NH₃ to give the amidine, b) converting the nitrile into the corresponding imidoester using an alcohol, for example ethanol, in the presence of HCl, and treating the imidoester with ammonia (Pinner synthesis), or c) reacting the nitrile with lithium bis(trimethylsilyl)amide, and subsequently hydrolysing the product.

Esters can be saponified, for example, using acetic acid or using NaOH or KOH in water, water/THF or water/dioxane, at temperatures between 0 and 100°.

Free amino groups can furthermore be acylated in a conventional manner using an acid chloride or anhydride or alkylated using an unsubstituted or substituted alkyl halide or reacted with CH₃—C(═NH)—OEt, advantageously in an inert solvent, such as dichloromethane or THF, and/or in the presence of a base, such as triethylamine or pyridine, at temperatures between −60 and +30°.

A base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable salts. Thus, it is possible to use inorganic acids, for example sulfuric acid, nitric acid, hydrohalic acids, such as hydrochloric acid or hydrobromic acid, phosphoric acids, such as orthophosphoric acid, sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalene-mono- and -disulfonic acids, laurylsulfuric acid. Salts with physiologically unacceptable acids, for example picrates, can be used for the isolation and/or purification of the compounds of the formula I.

On the other hand, compounds of the formula I can be converted into the corresponding metal salts, in particular alkali metal or alkaline earth metal salts, or into the corresponding ammonium salts using bases (for example sodium hydroxide, potassium hydroxide, sodium carbonate or potassium carbonate).

It is also possible to use physiologically acceptable organic bases, such as, for example, ethanolamine.

Compounds of the formula I according to the invention may be chiral owing to their molecular structure and may accordingly occur in various enantiomeric forms. They can therefore exist in racemic or in optically active form.

Since the pharmaceutical activity of the racemates or stereoisomers of the compounds according to the invention may differ, it may be desirable to use the enantiomers. In these cases, the end product or even the intermediates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitably N-protected amino acids (for example N-benzoylproline) or N-benzenesulfonylproline) or the various optically active camphorsulfonic acids. Also advantageous is chromatographic enantiomer resolution with the aid of an optically active resolving agent (for example dinitrobenzoylphenylglycine, cellulose triacetate or other derivatives of carbohydrates or chirally derivatised methacrylate polymers immobilised on silica gel). Suitable eluents for this purpose are aqueous or alcoholic solvent mixtures, such as, for example, hexane/isopropanol/acetonitrile, for example in the ratio 82:15:3.

The invention furthermore relates to the use of the compounds of the formula I and/or physiologically acceptable salts thereof for the preparation of pharmaceutical compositions, in particular by non-chemical methods. They can be converted here into a suitable dosage form together with at least one solid, liquid and/or semi-liquid excipient or adjuvant and, if desired, in combination with one or more further active ingredients.

The invention furthermore relates to medicaments comprising at least one compound of the formula I and/or pharmaceutically usable derivatives, solvates and stereoisomers thereof, including mixtures thereof in all ratios, and optionally excipients and/or adjuvants.

These compositions can be used in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatine, carbohydrates, such as lactose or starch, magnesium stearate, talc, Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders or also as nasal sprays. The novel compounds may also be lyophilised and the resultant lyophilisates used, for example, to prepare injection preparations. The compositions indicated may be sterilised and/or comprise adjuvants, such as lubricants, preservatives, stabilisers and/or wetting agents, emulsifying agents, salts for modifying the osmotic pressure, buffer substances, colorants and flavours and/or a plurality of further active ingredients, for example one or more vitamins.

The compounds of the formula I and physiologically acceptable salts thereof can be used for combating and preventing thromboembolic diseases, such as thromboses, myocardial infarction, arteriosclerosis, inflammation, apoplexy, angina pectoris, restenosis after angioplasty and claudicatio intermittens, migraine, tumours, tumour diseases and/or tumour metastases.

In general, the substances according to the invention are preferably administered in doses between about 1 and 500 mg, in particular between 5 and 100 mg, per dosage unit. The daily dose is preferably between about 0.02 and 10 mg/kg of body weight. However, the specific dose for each patient depends on a very wide variety of factors, for example on the efficacy of the specific compound employed, on the age, body weight, general state of health, sex, on the diet, on the time and method of administration, on the excretion rate, medicament combination and severity of the particular disease to which the therapy applies. Oral administration is preferred.

The invention also relates to a set (kit) consisting of separate packs of
(a) an effective amount of a compound of the formula I and/or pharmaceutically usable derivatives, solvates and stereoisomers thereof, including mixtures thereof in all ratios, and
(b) an effective amount of a further medicament.

The set comprises suitable containers, such as boxes, individual bottles, bags or ampoules. The set may, for example, comprise separate ampoules each containing an effective amount of a compound of the formula I and/or pharmaceutically usable derivatives, solvates and stereoisomers thereof, including mixtures thereof in all ratios,

and an effective amount of a further medicament in dissolved or lyophilised form.

The invention furthermore relates to the use of compounds of the formula I and/or pharmaceutically usable derivatives, solvates and stereoisomers thereof, including mixtures thereof in all ratios,

for the preparation of a medicament for the treatment of thromboses, myocardial infarction, arteriosclerosis, inflammation, apoplexy, angina pectoris, restenosis after angioplasty, claudicatio intermittens, migraine, tumours, tumour diseases and/or tumour metastases,

in combination with at least one further medicament active ingredient.

Above and below, all temperatures are given in ° C. In the following examples, “conventional work-up” means that water is added if necessary, the pH is adjusted, if necessary, to between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation. Rf values on silica gel; eluent: ethyl acetate/methanol 9:1.
Mass spectrometry (MS): EI (electron impact ionisation) M⁺
   FAB (fast atom bombardment) (M+H)⁺
   ESI (electrospray ionisation) (M+H)⁺ (unless stated otherwise)
      Preparation of Starting Materials

The aniline derivatives are prepared, for example, analogously to the following scheme: 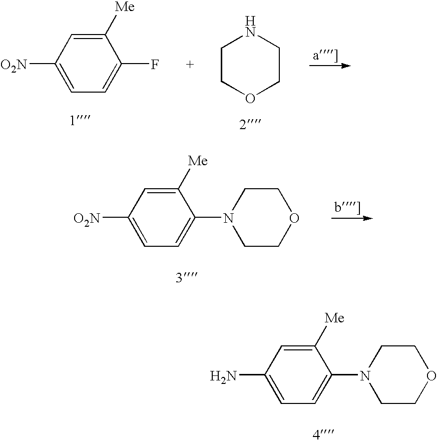
Unit 4′″:
Step a″″]

5.0 g (32.23 mmol) of 2-fluoro-5-nitrotoluene 1″″ and 1.93 ml (32.23 mmol) of morpholine 2″″ are dissolved in 30 ml of DMF, 10.5 g (32.23 mmol) of CsCO₃ are subsequently added, and the mixture is stirred at 110° C. for 18 hours. Conventional work-up thus gives 2.86 g (40%) of 4-(2-methyl-4-nitrophenyl)morpholine 3″″; melting point 154-155° C., MS [M+H]⁺ =223.

Step b″″]

2.75 g (12.374 mmol) of 3″″ are dissolved in 30 ml of THF, 1.0 g of 5% Pd/C(H₂O-moist) is subsequently added, and the mixture is stirred over an H₂ atmosphere until the theoretical amount of hydrogen has been taken up. Conventional work-up thus gives 2.35 g (98.8%) of 3-methyl-4-morpholin-4-ylphenylamine 4″″; melting point 83-84° C., MS [M+H]⁺ =193.

Analogously to this reaction sequence, 4-morpholin-4-yl-3-trifluoromethyl-phenylamine, 3-methyl-4-piperidin-1-ylphenylamine and 3-methyl-4-pyrrolidin-1-ylphenylamine, for example, are prepared.

The preparation of 1-(4-amino-2-methylphenyl)piperidin-2-one is also carried out, for example, as indicated below:

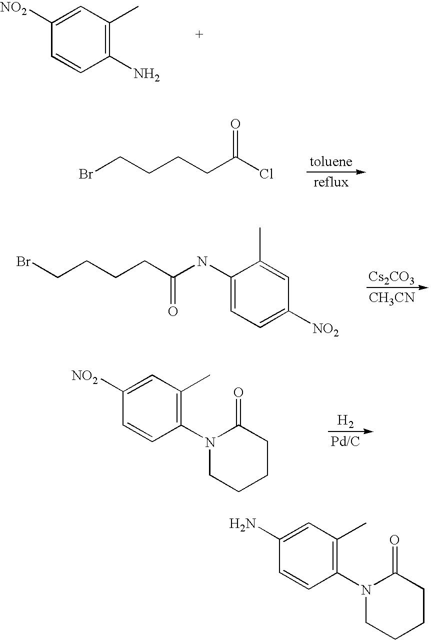

### EXAMPLE 1

The preparation of compounds of the following formula I-1 as listed in Table 1:

TABLE 1

| | |
|---|---|
| | I-1 |
| 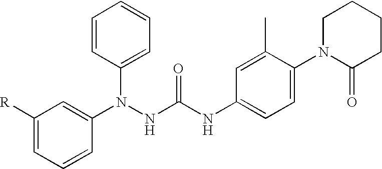 | |

| No. | R | Salt form |
|---|---|---|
| 6 | CN | |
| 8 | —C(═N—OH)—NH₂ | |
| 10 | C(═NH)—NH₂ | HCl |
| 12 | CH₂NH₂ | HCl |

and of compounds of the following formula I-2 as listed in Table 2:

TABLE 2

| | |
|---|---|
| | I-2 |
| 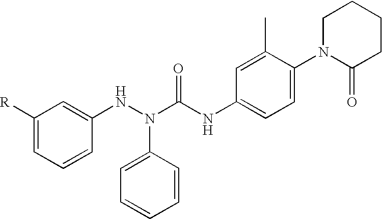 | |

| No. | R | Salt form |
|---|---|---|
| 7 | CN | |
| 9 | —C(═N—OH)—NH₂ | |
| 11 | C(═NH)—NH₂ | HCl |
| 13 | CH₂NH₂ | HCl |

is carried out analogously to the following reaction scheme: 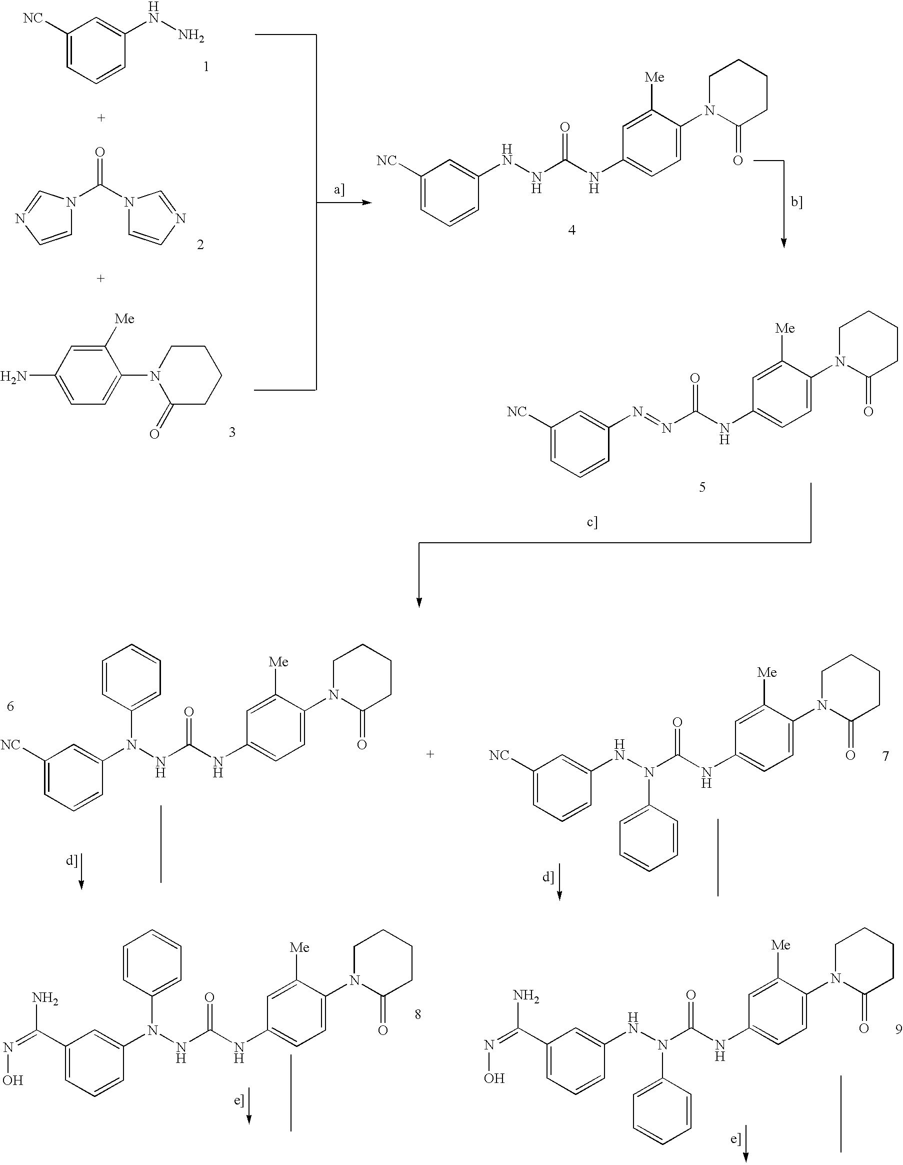 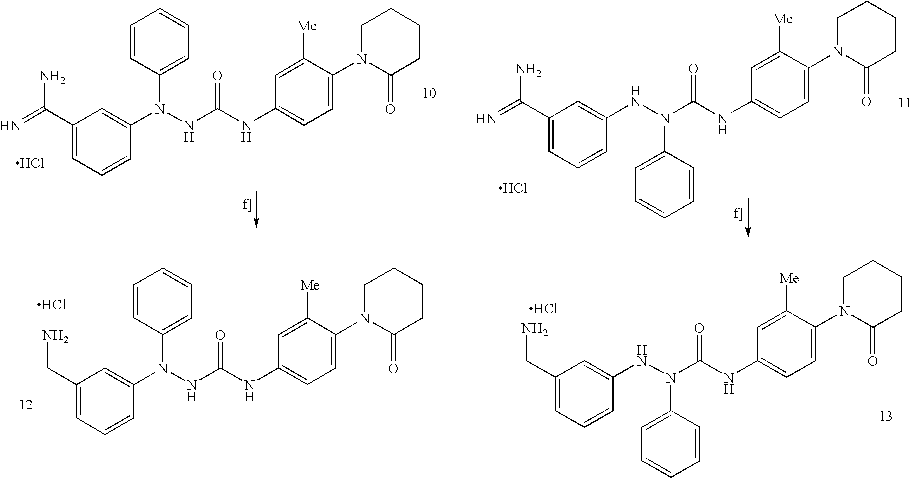
Step a]

3.0 g (22.53 mmol) of 3-hydrazinobenzonitrile 1 [preparation from 3-aminobenzonitrile: R. M. Acheson, J. M. Vernon J. Chem. Soc. 1962, 148-1157; O. Dann et al. Annalen 1975, 160-194] are dissolved in 100 ml of THF at 0° C. under a nitrogen atmosphere, and 3.654 g (22.53 mmol) of 1,1′-carbonyldiimidazole 2 are then added with stirring. The mixture is subsequently stirred at this temperature for 30 minutes. After the ice bath has been removed, 6.02 g (22.53 mmol) of 1-(4-amino-2-methylphenyl)piperidin-2-one 3 are added, and the mixture is stirred at RT for a further 1 hour. Conventional work-up thus gives 5.7 g (69.6%) of 1-(3-cyanophenyl)-4-[3-methyl-4-(2-oxopiperidin-1-yl)phenyl]semicarbazide 4; m.p. 206-207°, MS [M+H]⁺ 364.

Step b]

4.5 g (12.382 mmol) of 4 are dissolved in 30 ml of ethanol, 15 ml of water and 10 ml of hydrochloric acid (fuming, 37%) are added, and 2.135 g (30.955 mmol) of sodium nitrite (dissolved in 15 ml of water) are subsequently added dropwise at RT. The mixture is stirred at RT for 5 hours and subjected to conventional work-up, thus giving 4.2 g (93.9%) of diazene-carboxamide 5; m.p. 194-195°, MS [M+H]⁺ 362.

Two alternatives for this step: a. potassium chlorate, sulfuric acid, cat. Fe(II) sulfate, acetone or b. Cu(II) acetate, pyridine, dichloromethane.

Step c]

1.5 g (4.15 mmol) of 5 are dissolved in 50 ml of THF and cooled to −70°. 10.0 ml (10.0 mmol) of phenylmagnesium bromide (1 M in THF) are then added dropwise with stirring. Stirring is subsequently continued at this temperature for a further 30 minutes. Conventional work-up and chromatography on silica gel thus gives 1.3 g (71.3%) of 1-(3-cyanophenyl)-4-[3-methyl-4-(2-oxopiperidin-1-yl)phenyl]-1-phenylsemicarbazide (6), m.p. 161-162°, MS [M+H]⁺ 440;

and 0.42 g (23.0%) of 1-(3-cyanophenyl)-4-[3-methyl-4-(2-oxopiperidin-1-yl)phenyl]-2-phenylsemicarbazide 7, m.p. 136-137°, MS [M+H]⁺ 440.

If the reaction is carried out at −20°, the following yields are obtained under otherwise identical conditions: 0.61 g (33.4%) of 6 and 1.02 g (55.9%) of 7.

Step d]

0.4 g (0.91 mol) of 6 is dissolved in 15 ml of ethanol, 0.504 ml (3.64 mmol) of triethylamine and 0.253 g (3.64 mmol) of hydroxylammonium chloride are added, and the mixture is stirred at 75° C. for 3 hours. Conventional work-up thus gives 0.39 g (90.7%) of 1-(3-N-hydroxy-amidinophenyl)-4-[3-methyl-4-(2-oxopiperidin-1-yl)phenyl]-1-phenylsemicarbazide 8, m.p. 208-209°, MS [M+H]⁺ 474.

An analogous reaction of 7 gives 1-(3-N-hydroxyamidinophenyl)-4-[3-methyl-4-(2-oxopiperidin-1-yl)phenyl]-2-phenylsemicarbazide 9; m.p. 243-244°, MS [M+H]⁺ 474.

Step e]

0.15 g (0.317 mmol) of 8 is dissolved in 2 ml of methanol and 2 ml of THF, and 0.2 g of Raney nickel (50%), 2 ml of HOAc and 2 ml of water are added. The mixture is subsequently stirred overnight at atmospheric pressure and RT under an H₂ atmosphere. Conventional work-up thus gives 130 mg (89.9%) of 1-(3-amidinophenyl)-4-[3-methyl-4-(2-oxopiperidin-1-yl)phenyl]-1-phenylsemicarbazide (free base) 10.

The free base is dissolved in 5 ml of dichloromethane and 1 ml of methanol and stirred for 1 hour at RT with 2 ml of approximately 4N HCl in dioxane. Conventional work-up thus gives 140 mg (90%) of 1-(3-amidinophenyl)-4-[3-methyl-4-(2-oxopiperidin-1-yl)phenyl]-1-phenylsemicarbazide, hydrochloride 10, m.p. >300° C., MS [M+H]⁺ 457.

An analogous reaction of 9 gives 1-(3-amidinophenyl)-4-[3-methyl-4-(2-oxopiperidin-1-yl)phenyl]-2-phenylsemicarbazide, hydrochloride 11; m.p. >300°, MS [M+H]⁺ 457.

Step f]

0.5 g (1.138 mmol) of 6 is dissolved in 3 ml of methanol, and 0.3 g of Raney nickel (50%) and 2 ml of NH₃/methanol are added. The mixture is subsequently stirred overnight at 5 bar and 50° C. under an H₂ atmosphere. Conventional work-up thus gives 395 mg (78.3%) of 1-(3-aminomethylphenyl)-4-[3-methyl-4-(2-oxopiperidin-1-yl)phenyl]-1-phenylsemicarbazide (free base) 12.

The free base is dissolved in 5 ml of dichloromethane and stirred for 1 hour at RT with 2 ml of approximately 4 N HCl in dioxane. Conventional work-up thus gives 400 mg (73.2%) of 1-(3-aminomethylphenyl)-4-[3-methyl-4-(2-oxopiperidin-1-yl)phenyl]-1-phenylsemicarbazide, hydrochloride 12; m.p. 172-173°, MS [M+H]⁺ 444.

An analogous reaction of 7 gives 1-(3-aminomethylphenyl)-4-[3-methyl-4-(2-oxopiperidin-1-yl)phenyl]-2-phenylsemicarbazide, hydrochloride 13; m.p. 95-97°, MS [M+H]⁺ 444.

### EXAMPLE 2

### 1-(3-Cyanophenyl)-4-[3-methyl-4-(morpholin-4-yl)phenyl]-1-phenylsemicarbazide (5′)

The preparation is carried out analogously to the following scheme: 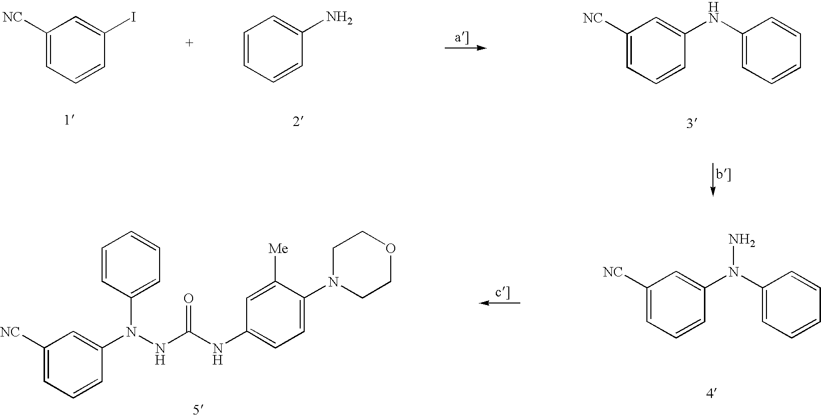
Step a′]

Analogously to J. P. Wolfe et al. J. Org. Chem. 2000, 65, 1158-1174: 0.46 g (0.005 mol) of Pd₂(dba)₃ (dba=dibenzylideneacetone), 0.7 (0.02 mol) of biphenyl-2-yldicyclohexylphosphine and 29.7 g (0.14 mol) of potassium phosphate are dissolved in 1000 ml of DME at RT under an inert gas and with stirring. 22.2 g (0.1 mol) of 3-iodobenzoic acid 1′ and 11.18 g (0.12 mol) of aniline 2′ are then added, and the mixture is subsequently stirred at 100° C. After 24 hours, the mixture is subjected to conventional work-up, thus giving 3-phenylaminobenzonitrile 3′, MS [M+H]⁺ 195.

Step b′]

A solution of 0.72 g (0.011 mol) of sodium nitrite in ml of water is added dropwise to a solution of 1.943 g (0.01 mol) of 3′ in 1.5 ml of conc. HCl and 6 g of crushed ice at such a rate that the temperature does not exceed 5° C. After 2 hours, the mixture is subjected to conventional work-up, thus giving the N-nitroso derivative of 3-(N-phenylhydrazino)benzonitrile 4′, which is further reacted directly. N-Nitroso-4′ in 5 ml of glacial acetic acid is added dropwise to a suspension of 3.0 g of zinc dust in 5 ml of water at such a rate that the temperature remains between 5 and 10° C. The mixture is then stirred for 1 hour at RT and then for 2 hours at 80° C. Conventional work-up thus gives 3-(N-phenylhydrazino)benzonitrile 4′, MS (M⁺) 210.

Step c′]

This step is carried out analogously to step a] with 4′ and 3-methyl-4-morpholin-4-ylphenylamine 4″″, thus giving 1-(3-cyanophenyl)-4-[3-methyl-4-(morpholin-4-yl)phenyl]-1-phenylsemicarbazide 5′, m.p. 115-116°, MS [M+H]⁺ 428.

### EXAMPLE 3

### 1-(3-Cyanophenyl)-4-[4-(2-oxopiperidin-1-yl)phenyl]-2-phenylsemicarbazide (5″)

The preparation is carried out analogously to the following scheme: 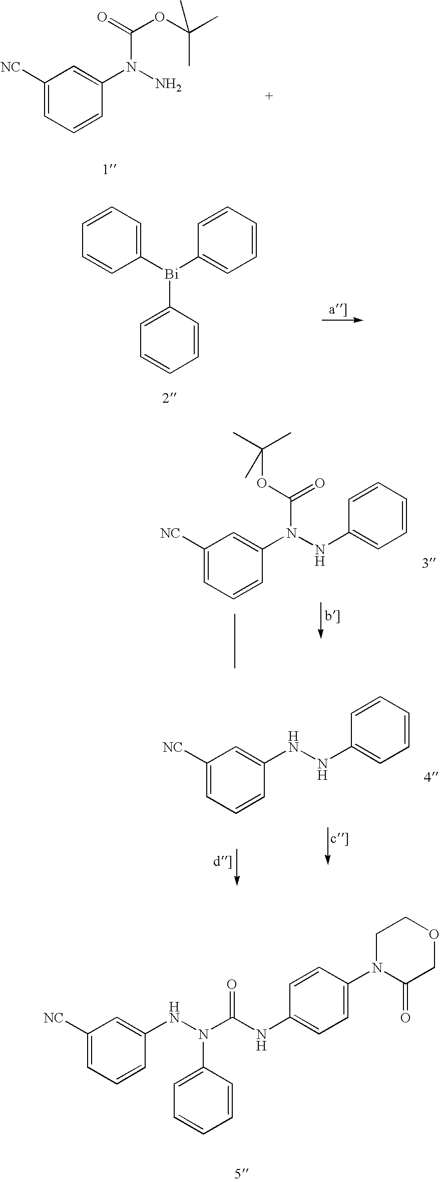
Step a″]

Analogously to DHR Barton et al. Tet. Lett. 1987, 28, 887: 5.0 g (21.434 mmol) of tert-butyl N-(3-cyanophenyl)hydrazinecarboxylate 1″, 9.44 g (21.434 mmol) of triphenylbismuth 2″ and 4.87 g (26.79 mmol) of Cu(II) acetate are dissolved in 60 ml of DCM and stirred at 40° C. for 18 hours. Conventional work-up thus gives 3.21 g (48.4%) of tert-butyl N-(3-cyanophenyl)-N′-phenylhydrazinecarboxylate 3″, MS [M+H]⁺ 310.

Step b″]

0.5 g (1.616 mmol) of 3″ is heated at 150° for 5 hours. Conventional work-up thus gives 0.32 g (94.6%) of 3-(N′-phenylhydrazino)benzonitrile 4″, MS [M+H]⁺ 210.

Step c″] and step d″]

Both reactions are carried out analogously to step a] either with 3″ or 4″ and 1-(4-aminophenyl)piperidin-2-one, thus giving in both cases (in the case of d″] in situ removal of the Boc group) 1-(3-cyanophenyl)-4-[4-(2-oxopiperidin-1-yl)phenyl]-2-phenylsemicarbazide 5″, m.p. 239-241°; MS [M+H]⁺ 426.

These “urea formation reactions” can also be carried out analogously thereto with the corresponding isocyanates (in situ preparation) instead of with carbonyldiimidazole. An example in this respect is given in the literature, where 1,2,4-triphenylsemicarbazide has been prepared by reaction of hydrazobenzene and phenyl isocyanate within 2 days at RT in benzene; D. Sarantakis et al. Tet. Lett. 1987, 2578.

### EXAMPLE 4

The following compounds are obtained analogously to Example 1:
1-(3-amidinophenyl)-4-[3-methyl-4-(morpholin-4-yl)phenyl]-1-phenylsemicarbazide, hydrochloride;
1-(3-amidinophenyl)-4-[3-methyl-4-(2-oxopiperidin-1-yl)phenyl]-2-phenylsemicarbazide, hydrochloride;
1-(3-amidinophenyl)-4-[4-(2-oxopiperidin-1-yl)phenyl]-1-phenylsemicarbazide, hydrochloride;
1-(3-amidinophenyl)-4-[4-(2-oxopiperidin-1-yl)phenyl]-2-phenylsemicarbazide, hydrochloride;
1-(3-amidinophenyl)-4-[3-chloro-4-(3-oxomorpholin-4-yl)phenyl]-1-phenylsemicarbazide, hydrochloride;
1-(3-amidinophenyl)-4-[3-chloro-4-(3-oxomorpholin-4-yl)phenyl]-2-phenylsemicarbazide, hydrochloride (“4.AA”);
1-(3-aminomethylphenyl)-4-[3-methyl-4-(morpholin-4-yl)phenyl]-1-phenylsemicarbazide, hydrochloride;
1-(3-aminomethylphenyl)-4-[3-methyl-4-(2-oxopiperidin-1-yl)phenyl]-2-phenylsemicarbazide, hydrochloride;
1-(3-aminomethylphenyl)-4-[4-(2-oxopiperidin-1-yl)phenyl]-1-phenylsemicarbazide, hydrochloride;
1-(3-aminomethylphenyl)-4-[4-(2-oxopiperidin-1-yl)phenyl]-2-phenylsemicarbazide, hydrochloride;
1-(3-aminomethylphenyl)-4-[3-chloro-4-(3-oxomorpholin-4-yl)phenyl]-1-phenylsemicarbazide, hydrochloride;
1-(3-aminomethylphenyl)-4-[3-chloro-4-(3-oxomorpholin-4-yl)phenyl]-2-phenylsemicarbazide, hydrochloride;
1-(3-aminomethylphenyl)-4-[3-chloro-4-(morpholin-4-yl)phenyl]-1-phenylsemicarbazide, hydrochloride;
1-(3-aminomethylphenyl)-4-[3-chloro-4-(morpholin-4-yl)phenyl]-2-phenylsemicarbazide, hydrochloride;
1-(3-aminomethylphenyl)-4-[3-methyl-4-(piperidin-1-yl)phenyl]-1-phenylsemicarbazide, hydrochloride;
1-(3-aminomethylphenyl)-4-[3-methyl-4-(piperidin-1-yl)phenyl]-2-phenylsemicarbazide, hydrochloride;
1-(3-aminomethylphenyl)-4-[3-chloro-4-(piperidin-1-yl)phenyl]-1-phenylsemicarbazide, hydrochloride;
1-(3-aminomethylphenyl)-4-[3-chloro-4-(piperidin-1-yl)phenyl]-2-phenylsemicarbazide, hydrochloride;
1-(3-aminomethylphenyl)-4-[4-(2-oxo-1H-pyridin-1-yl)phenyl]-1-phenylsemicarbazide, hydrochloride;
1-(3-aminomethylphenyl)-4-[4-(2-oxo-1H-pyridin-1-yl)phenyl]-2-phenylsemicarbazide, hydrochloride;
1-(3-aminomethylphenyl)-4-[4-(2-oxopiperidin-1-yl)phenyl]-1-(4-fluorophenyl)semicarbazide, hydrochloride;
1-(3-aminomethylphenyl)-4-[4-(2-oxopiperidin-1-yl)phenyl]-2-(4-fluorophenyl)semicarbazide, hydrochloride;
1-(3-aminomethylphenyl)-4-[3-methyl-4-(2-oxopiperidin-1-yl)phenyl]-1-(4-fluorophenyl)semicarbazide, hydrochloride;
1-(3-aminomethyl phenyl)-4-[3-methyl-4-(2-oxopiperidin-1-yl)phenyl]-2-(4-fluorophenyl)semicarbazide, hydrochloride;
1-(3-aminomethylphenyl)-4-[3-methyl-4-(3-oxomorpholin-4-yl)phenyl]-1-(4-fluorophenyl)semicarbazide, hydrochloride;
1-(3-aminomethylphenyl)-4-[3-methyl-4-(3-oxomorpholin-4-yl)phenyl]-2-(4-fluorophenyl)semicarbazide, hydrochloride;
1-(3-aminomethylphenyl)-4-[4-(3-oxo-2H-pyridazin-2-yl)phenyl]-1-phenylsemicarbazide, hydrochloride;
1-(3-aminomethylphenyl)-4-[4-(3-oxo-2H-pyridazin-2-yl)phenyl]-2-phenylsemicarbazide, hydrochloride;
1-(3-aminomethylphenyl)-4-[4-(2-oxopyrrolidin-1-yl)phenyl]-1-(4-fluorophenyl)semicarbazide, hydrochloride;
1-(3-aminomethylphenyl)-4-[4-(2-oxopyrrolidin-1-yl)phenyl]-2-(4-fluorophenyl)semicarbazide, hydrochloride;
1-(3-aminomethylphenyl)-4-[3-methyl-4-(2-oxopyrrolidin-1-yl)phenyl]-1-phenylsemicarbazide, hydrochloride;
1-(3-aminomethylphenyl)-4-[3-methyl-4-(2-oxopyrrolidin-1-yl)phenyl]-2-phenylsemicarbazide, hydrochloride;
1-(3-aminomethylphenyl)-4-[4-(3-oxo-2H-piperazin-1-yl)phenyl]-1-phenylsemicarbazide, hydrochloride;
1-(3-aminomethylphenyl)-4-[4-(3-oxo-2H-piperazin-1-yl)phenyl]-2-phenylsemicarbazide, hydrochloride;
1-(3-aminomethylphenyl)-4-[4-(2-oxo-1,3-oxazolidin-3-yl)phenyl]-1-phenylsemicarbazide, hydrochloride;
1-(3-aminomethylphenyl)-4-[4-(2-oxo-1,3-oxazolidin-3-yl)phenyl]-2-phenylsemicarbazide, hydrochloride;
1-(3-aminomethylphenyl)-4-[4-(2-caprolactam-1-yl)phenyl]-1-phenylsemicarbazide, hydrochloride;
1-(3-aminomethylphenyl)-4-[4-(2-caprolactam-1-yl)phenyl]-2-phenylsemicarbazide, hydrochloride;
1-(3-aminomethylphenyl)-4-[4-(3-oxo-2-azabicyclo[2.2.2]oct-2-yl)phenyl]-1-phenylsemicarbazide, hydrochloride;
1-(3-aminomethylphenyl)-4-[4-(3-oxo-2-azabicyclo[2.2.2]oct-2-yl)phenyl]-2-phenylsemicarbazide, hydrochloride;
1-(3-aminomethylphenyl)-4-[3-trifluoromethyl-4-(2-oxopiperidin-1-yl)phenyl]-1-phenylsemicarbazide, hydrochloride;
1-(3-aminomethylphenyl)-4-[3-trifluoromethyl-4-(3-oxomorpholin-4-yl)phenyl]-1-phenylsemicarbazide, hydrochloride;
1-(3-aminomethylphenyl)-4-[3-trifluoromethyl-4-(2-oxopiperidin-1-yl)phenyl]-1-(2-chlorophenyl)semicarbazide, hydrochloride,
1-(3-aminomethylphenyl)-4-[3-trifluoromethyl-4-(3-oxomorpholin-4-yl)phenyl]-1-(2-chlorophenyl)semicarbazide, hydrochloride;
1-(3-aminomethylphenyl)-4-[3-trifluoromethyl-4-(2-oxopiperidin-1-yl)phenyl]-2-phenylsemicarbazide, hydrochloride;
1-(3-aminomethyl phenyl)-4-[3-trifluoromethyl-4-(3-oxomorpholin-4-yl)phenyl]-2-phenylsemicarbazide, hydrochloride;
1-(3-aminomethylphenyl)-4-[3-trifluoromethyl-4-(2-oxopiperidin-1-yl)phenyl]-2-(2-chlorophenyl)semicarbazide, hydrochloride,
1-(3-aminomethylphenyl)-4-[3-trifluoromethyl-4-(3-oxomorpholin-4-yl)phenyl]-2-(2-chlorophenyl)semicarbazide, hydrochloride.
   Pharmacological Data

Affinity to Receptors

TABLE 3

| Compound | | |
|---|---|---|
| No. | FXa-IC₅₀ [M] | TF/FVIIa-IC₅₀ [M] |
| 10 | 16.0 E−9 | 9.8 E−9 |
| 11 | 8.6 E−9 | 5.1 E−9 |
| 13 | 130 E−9 | 170 E−9 |
| 4.AA | 20.0 E−9 | 27.0 E−9 |

The following examples relate to pharmaceutical compositions:

### EXAMPLE A

### Injection Vials

A solution of 100 g of an active ingredient of the formula I and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### EXAMPLE B

### Suppositories

A mixture of 20 g of an active ingredient of the formula I with 100 g of soya lecithin and 1400 g of cocoa butter is melted, poured into moulds and allowed to cool. Each suppository contains 20 mg of active ingredient.

### EXAMPLE C

### Solution

A solution is prepared from 1 g of an active ingredient of the formula I, 9.38 g of NaH₂PO₄ 2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient of the formula I are mixed with 99.5 g of Vaseline under aseptic conditions.

### EXAMPLE E

### Tablets

A mixture of 1 kg of active ingredient of the formula I , 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### EXAMPLE F

### Coated Tablets

Tablets are pressed analogously to Example E and subsequently coated in a conventional manner with a coating of sucrose, potato starch, talc, tragacanth and dye.

### EXAMPLE G

### Capsules

2 kg of active ingredient of the formula I are introduced into hard gelatine capsules in a conventional manner in such a way that each capsule contains 20 mg of the active ingredient.

### EXAMPLE H

### Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

1. Compounds of the formula I 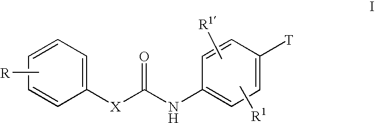
in which
R denotes C(═NH)—NH₂, which may also be monosubstituted by OH, COA or OCOOA,
or is CH₂NH₂, CONH₂, CN, 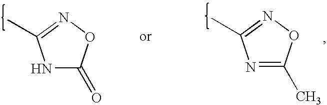
X denotes —N(Ar)—NH— or —NH—N(Ar)—,
R¹ and
R^{1′} each, independently of one another, denote H, A, OH, OA, —O(C═O)A, Hal, CF₃, CN, COOH, COOA, CH₂NH₂, NH₂, NHA or NA₂,
T denotes a monocyclic or bicyclic saturated or unsaturated heterocyclic ring having from 1 to 4 N, O and/or S atoms, which may be mono-, di- or trisubstituted by carbonyl oxygen and/or OH, Hal or A,
Ar denotes phenyl which is unsubstituted or mono-, di- or trisubstituted by A, OH, OA, —O—(C═O)-A, Hal, NH₂, NHA, NA₂, NO₂, CF₃, CN, COA, COOH, COOA, CONH₂ or CH₂NH₂,
A denotes unbranched, branched or cyclic alkyl having 1-10 carbon atoms,
Hal denotes F, Cl, Br or I,
and pharmaceutically usable derivatives, solvates and stereoisomers thereof, including mixtures thereof in all ratios.

2. Compounds according to claim 1, in which
R denotes amidino, which may also be substituted by OH, or denotes CH₂NH₂,
and pharmaceutically usable derivatives, solvates and stereoisomers thereof, including mixtures thereof in all ratios.

3. Compounds according to claim 1, in which
R¹ denotes H, A, CF₃ or Hal, and
R^{1″} denotes H,
and pharmaceutically usable derivatives, solvates and stereoisomers thereof, including mixtures thereof in all ratios.

4. Compounds according to claim 1, in which
Ar denotes phenyl which is unsubstituted or monosubstituted by Cl or F,
and pharmaceutically usable derivatives, solvates and stereoisomers thereof, including mixtures thereof in all ratios.

5. Compounds according to claim 1, in which
T denotes a monocyclic or bicyclic saturated or unsaturated heterocyclic ring having 1 or 2 N and/or O atoms, which may be monosubstituted or disubstituted by carbonyl oxygen and/or OH, Hal or A,
and pharmaceutically usable derivatives, solvates and stereoisomers thereof, including mixtures thereof in all ratios.

6. Compounds according to claim 1, in which
T denotes a monocyclic saturated or unsaturated heterocyclic ring having 1 to 2 N and/or O atoms, which may be monosubstituted or disubstituted by carbonyl oxygen,
and pharmaceutically usable derivatives, solvates and stereoisomers thereof, including mixtures thereof in all ratios.

7. Compounds according to claim 1, in which
T denotes piperidin-1-yl, 2-oxopiperidin-1-yl, pyrrolidin-1-yl, 2-oxopyrrolidin-1-yl, 2-oxo-1H-pyridin-1-yl, 3-oxomorpholin-4-yl, morpholin-4-yl, 4-oxo-1H-pyridin-1-yl, 2,6-dioxopiperidin 1-yl, 2-oxopiperazin-1-yl, 2,6-dioxopiperazin-1-yl, 2,5-dioxopyrrolidin-1-yl, 2-oxo-1,3-oxazolidin-3-yl, 3-oxo-2H-pyridazin-2-yl, 2-caprolactam-1-yl (=2-oxoazepan-1-yl), 2-hydroxy-6-oxopiperazin-1-yl, 2-azabicyclo[2.2.2]octan-3-on-2-yl, 2-methoxy-6-oxopiperazin-1-yl, 5,6-dihydro-1H-pyrimidin-2-oxo-1-yl or 4H-1,4-oxazin-4-yl,
and pharmaceutically usable derivatives, solvates and stereoisomers thereof, including mixtures thereof in all ratios.

8. Compounds according to claim 1, in which
T denotes 2-oxopiperidin-1-yl, 2-oxopyrrolidin-1-yl, 3-oxomorpholin-4-yl, morpholin-4-yl, piperidin-1-yl, pyrrolidin-1-yl or 3-oxo-2H-pyridazin-2-yl,
and pharmaceutically usable derivatives, solvates and stereoisomers thereof, including mixtures thereof in all ratios.

9. Compounds according to claim 1, selected from the group consisting of
1-(3-cyanophenyl)-4-[3-methyl-4-(2-oxopiperidin-1-yl)phenyl]-1-phenylsemicarbazide,
1-(3-cyanophenyl)-4-[3-methyl-4-(2-oxopiperidin-1-yl)phenyl]-2-phenylsemicarbazide,
1-(3-N-hydroxyamidinophenyl)-4-[3-methyl-4-(2-oxopiperidin-1-yl)phenyl]-1-phenylsemicarbazide,
1-(3-N-hydroxyamidinophenyl-4-[3-methyl-4-(2-oxopiperidin-1-yl)phenyl]-2-phenylsemicarbazide,
1-(3-amidinophenyl)-4-[3-methyl-4-(2-oxopiperidin-1-yl)phenyl]-1-phenylsemicarbazide,
1-(3-amidinophenyl)-4-[3-methyl-4-(2-oxopiperidin-1-yl)phenyl]-2-phenylsemicarbazide,
1-(3-aminomethylphenyl)-4-[3-methyl-4-(2-oxopiperidin-1-yl)phenyl]-1-phenylsemicarbazide,
1-(3-aminomethylphenyl-4-[3-methyl-4-(2-oxopiperidin-1-yl)phenyl]-2-phenylsemicarbazide,
1-(3-cyanophenyl)-4-[3-methyl-4-(morpholin-4-yl)phenyl]-1-phenylsemicarbazide,
1-(3-cyanophenyl)-4-[4-(2-oxopiperidin-1-yl)phenyl]-2-phenylsemicarbazide,
1-(3-amidinophenyl)-4-[3-methyl-4-(morpholin-4-yl)phenyl]-1-phenylsemicarbazide,
1-(3-amidinophenyl)-4-[3-methyl-4-(2-oxopiperidin-1-yl)phenyl]-2-phenylsemicarbazide,
1-(3-amidinophenyl)-4-[4-(2-oxopiperidin-1-yl)phenyl]-1-phenylsemicarbazide,
1-(3-amidinophenyl)-4-[4-(2-oxopiperidin-1-yl)phenyl]-2-phenylsemicarbazide,
1-(3-amidinophenyl)-4-[3-chloro-4-(3-oxomorpholin-4-yl)phenyl]-1phenylsemicarbazide,
1-(3-amidinophenyl)-4-[3-chloro-4-(3-oxomorpholin-4-yl)phenyl]-2-phenylsemicarbazide,
1-(3-aminomethylphenyl)-4-[3-methyl-4-(morpholin-4-yl)phenyl]-1-phenylsemicarbazide,
1-(3-aminomethylphenyl)-4-[3-methyl-4-(2-oxopiperidin-1-yl)phenyl]-2-phenylsemicarbazide,
1-(3-aminomethylphenyl)-4-[4-(2-oxopiperidin-1-yl)phenyl]-1-phenylsemicarbazide,
1-(3-aminomethylphenyl)-4-[4-(2-oxopiperidin-1-yl)phenyl]-2-phenylsemicarbazide,
1-(3-aminomethylphenyl)-4-[3-chloro-4-(3-oxomorpholin-4-yl)phenyl]-1-phenylsemicarbazide,
1-(3-aminomethylphenyl)-4-[3-chloro-4-(3-oxomorpholin-4-yl)phenyl]-2-phenylsemicarbazide,
1-(3-aminomethylphenyl)-4-[3-chloro-4-(morpholin-4-yl)phenyl]-1-phenylsemicarbazide,
1-(3-aminomethylphenyl)-4-[3-chloro-4-(morpholin-4-yl)phenyl]-2-phenylsemicarbazide,
1-(3-aminomethylphenyl)-4-[3-methyl-4-(piperidin-1-yl)phenyl]-1-phenylsemicarbazide,
1-(3-aminomethylphenyl)-4-[3-methyl-4-(piperidin-1-yl)phenyl]-2-phenylsemicarbazide,
1-(3-aminomethylphenyl)-4-[3-chloro-4-(piperidin-1-yl)phenyl]-1-phenylsemicarbazide,
1-(3-aminomethylphenyl)-4-[3-chloro-4-(piperidin-1-yl)phenyl]-2-phenylsemicarbazide,
1-(3-aminomethylphenyl)-4-[4-(2-oxo-1H-pyridin-1-yl)phenyl]-1-phenylsemicarbazide,
1-(3-aminomethylphenyl)-4-[4-(2-oxo-1H-pyridin-1-yl)phenyl]-2-phenylsemicarbazide,
1-(3-aminomethylphenyl)-4-[4-(2-oxopiperidin-1-yl)phenyl]-1-(4-fluorophenyl)semicarbazide,
1-(3-aminomethylphenyl)-4-[4-(2-oxopiperidin-1-yl)phenyl]-2-(4-fluorophenyl)semicarbazide,
1-(3-aminomethylphenyl)-4-[3-methyl-4-(2-oxopiperidin-1-yl)phenyl]-1-(4-fluorophenyl)semicarbazide,
1-(3-aminomethylphenyl)-4-[3-methyl-4-(2-oxopiperidin-1-yl)phenyl]-2-(4-fluorophenyl)semicarbazide,
1-(3-aminomethylphenyl)-4-[3-methyl-4-(3-oxomorpholin-4-yl)phenyl]-1-(4-fluorophenyl)semicarbazide,
1-(3-aminomethylphenyl)-4-[3-methyl-4-(3-oxomorpholin-4-yl)phenyl]-2-(4-fluorophenyl)semicarbazide,
1-(3-aminomethylphenyl)-4-[4-(3-oxo-2H-pyridazin-2-yl)phenyl]-1-phenylsemicarbazide,
1-(3-aminomethylphenyl)-4-[4-(3-oxo-2H-pyridazin-2-yl)phenyl]-2-phenylsemicarbazide,
1-(3-aminomethylphenyl)-4-[4-(2-oxopyrrolidin-1-yl)phenyl]-1-(4-fluorophenyl)semicarbazide,
1-(3-aminomethylphenyl)-4-[4-(2-oxopyrrolidin-1-yl)phenyl]-2-(4-fluorophenyl)semicarbazide,
1-(3-aminomethylphenyl)-4-[3-methyl-4-(2-oxopyrrolidin-1-yl)phenyl]-1-phenylsemicarbazide,
1-(3-aminomethylphenyl)-4-[3-methyl-4-(2-oxopyrrolidin-1-yl)phenyl]-2-phenylsemicarbazide,
1-(3-aminomethylphenyl)-4-[4-(3-oxo-2H-piperazin-1-yl)phenyl]-1-phenylsemicarbazide,
1-(3-aminomethylphenyl)-4-[4-(3-oxo-2H-piperazin-1-yl)phenyl]-2-phenylsemicarbazide,
1-(3-aminomethylphenyl)-4-[4-(2-oxo-1,3-oxazolidin-3-yl)phenyl]-1-phenylsemicarbazide,
1-(3-aminomethylphenyl)-4-[4-(2-oxo-1,3-oxazolidin-3-yl)phenyl]-2phenylsemicarbazide,
1-(3-aminomethylphenyl)-4-[4-(2-caprolactam-1-yl)phenyl]-1-phenylsemicarbazide,
1-(3-aminomethylphenyl)-4-[4-(2-caprolactam-1-yl)phenyl]-2-phenylsemicarbazide,
1-(3-aminomethylphenyl)-4-[4-(3-oxo-2-azabicyclo[2.2.2]oct-2-yl)phenyl]-1-phenylsemicarbazide,
1-(3-aminomethylphenyl)-4-[4-(3-oxo-2-azabicyclo[2.2.2]oct-2-yl)phenyl]-2-phenylsemicarbazide,
1-(3-aminomethylphenyl)-4-[3-trifluoromethyl-4-(2-oxopiperidin-1-yl)phenyl]-1-phenylsemicarbazide,
1-(3-aminomethylphenyl)-4-[3-trifluoromethyl-4-(3-oxomorpholin-4-yl)phenyl]-1-phenylsemicarbazide,
1-(3-aminomethylphenyl)-4-[3-trifluoromethyl-4-(2-oxopiperidin-1-yl)phenyl]-1-(2-chlorophenyl)semicarbazide,
1-(3-aminomethylphenyl)-4-[3-trifluoromethyl-4-(3-oxomorpholin-4-yl)phenyl]-1-(2-chlorophenyl)semicarbazide,
1-(3-aminomethylphenyl)-4-[3-trifluoromethyl-4-(2-oxopiperidin-1-yl)phenyl]-2-phenylsemicarbazide,
1-(3-aminomethylphenyl)-4-[3-trifluoromethyl-4-(3-oxomorpholin-4-yl)phenyl]-2-phenylsemicarbazide,
1-(3-aminomethylphenyl)-4-[3-trifluoromethyl-4-(2-oxopiperidin-1-yl)phenyl]-2-(2-chlorophenyl)semicarbazide,
1-(3-aminomethylphenyl)-4-[3-trifluoromethyl-4-(3-oxomorpholin-4-yl)phenyl]-2-(2-chlorophenyl)semicarbazide,
and pharmaceutically usable derivatives, solvates and stereoisomers thereof, including mixtures thereof in all ratios.

10. Process for the preparation of compounds of the formula I according to claim 1 and pharmaceutically usable derivatives, solvates and stereoisomers thereof, characterised in that
a) they are liberated from one of their functional derivatives by treatment with a solvolysing and/or hydrogenolysing agent by
i) liberating an amidino group from the oxadiazole derivative or oxazolidinone derivative thereof by hydrogenolysis or solvolysis,
ii) replacing a conventional amino-protecting group with hydrogen by treatment with a solvolysing or hydrogenolysing agent or liberating an amino group protected by a conventional protecting group,
b) a radical R is converted into another radical R by
i) converting a cyano group into an amidino group,
ii) reducing a cyano group to an aminoalkyl group,
and/or
a base or acid of the formula I is converted into one of its salts.

11. Compounds of the formula I according to claim 1 as inhibitors of coagulation factor Xa.

12. Compounds of the formula I according to claim 1 as inhibitors of coagulation factor VIIa.

13. Medicaments comprising at least one compound of the formula I according to claim 1 and/or pharmaceutically usable derivatives, solvates and stereoisomers thereof, including mixtures thereof in all ratios, and optionally excipients and/or adjuvants.

14. Medicaments comprising at least one compound of the formula I according to claim 1 and/or pharmaceutically usable derivatives, solvates and stereoisomers thereof, including mixtures thereof in all ratios, and at least one further medicament active ingredient.

15. Use of compounds according to claim 1 and/or physiologically acceptable salts and solvates thereof for the preparation of a medicament for the treatment of thromboses, myocardial infarction, arteriosclerosis, inflammation, apoplexy, angina pectoris, restenosis after angioplasty, claudicatio intermittens, migraine, tumours, tumour diseases and/or tumour metastases.

16. Set (kit) consisting of separate packs of
(a) an effective amount of a compound of the formula I according to claim 1 and/or pharmaceutically usable derivatives, solvates and stereoisomers thereof, including mixtures thereof in all ratios, and
(b) an effective amount of a further medicament active ingredient.

17. Use of compounds of the formula I according to claim 1 and/or pharmaceutically usable derivatives, solvates and stereoisomers thereof, including mixtures thereof in all ratios, for the preparation of a medicament for the treatment of thromboses, myocardial infarction, arteriosclerosis, inflammation, apoplexy, angina pectoris, restenosis after angioplasty, claudicatio intermittens, migraine, tumours, tumour diseases and/or tumour metastases, in combination with at least one further medicament active ingredient.
